(19) United States
(12) Patent Application Publication
(10) Pub. No.: US 2019/0060243 A1
(43) Pub. Date: Feb. 28, 2019

(54) ARTICLE AND METHOD FOR A PHARMACEUTICAL AGENT

(71) Applicant: MD Medical Research, Oxon Hill, MD (US)

(72) Inventors: Spencer ONG, Oxon Hill, MD (US); Stephen ONG, Oxon Hill, MD (US)

(21) Appl. No.: 15/969,276

(22) Filed: May 2, 2018

Related U.S. Application Data
(60) Provisional application No. 62/550,324, filed on Aug. 25, 2017.

(51) Int. Cl. A61K 9/20

(52) U.S. Cl. CPC A61K 9/2072; A61K 9/2095

(57) ABSTRACT

A versatile and effective manner to provide a single tablet or tablet arrangement or other medication delivery system for a combination of medications comprising of two or more active ingredients to be administered by, for example, severing along the score line to separate the active ingredients and to consume each active ingredient separately or in various ratios depending on the applicable pharmaceutical agent that a patient desires to ingest or otherwise administer. In some embodiments, a single tablet comprising of two or more active ingredients is administered by severing along score lines to separate the active ingredients to consume two or more active ingredients in combination. This delivery system provides a variety of alternatives to a consumer for administrating a combination of active ingredients while varying the relevant rations of the active ingredients. Simultaneous administration of medication is also provided by specific inhaler and syringe systems.

### CLAIM FOR PRIORITY

The present application claims priority from U.S. Provisional Patent Application Ser. No. 62/550,324, filed Aug. 25, 2017, which is hereby incorporated by reference in its entirety.

### BACKGROUND OF THE INVENTION

### Field of Invention

This invention relates to products, methods and systems for pharmaceutical formulations having two or more pharmaceutical compositions where the formulation and administration of varying ratios of such compositions may be enhanced.

### Description of the Related Art

It is well-known to produce pharmaceutical pills with fracture scores that permit dosage forms to break into smaller portions, thus making it possible to evaluate multiple dosing regimens. This characteristic can be useful because the score can be used to facilitate the splitting of the tablet into fractions when less than a full tablet is desired for a dose.

A drug manufacturer may score pills to indicate that a pill may be split and to aid in the practice of splitting pills by the consumer. However, when a drug manufacturer chooses to do so, it must be consistent for consumers to be able to use them effectively.

It is also well-known that various combinations of medication is administered at the prescription of a doctor. However, many patients are not compliant with their combination of medications as prescribed because it is inconvenient to take more than one medication, and oftentimes it is difficult for patients to remember when to take which medication, since some are required at different times during the day, or with or without food.

By way of example, the prevalence of obesity is increasing worldwide. In 2010, the International Obesity Taskforce estimated that approximately 1 billion adults were overweight and more than 500 million were obese. In the United States, from 2011 to 2012, 32.9% of adults and 16.9% of children and adolescents were obese. The percentage of overweight adults was 68.5%. The United States Center for Disease Control and Prevention predict that obesity-related deaths could be a leading cause of mortality in the country. Obesity and being overweight are major risk factors for chronic cardiovascular disease, type 2 diabetes mellitus, hypertension, and certain types of cancers. While this disclosure uses obesity and diabetes as an example of one ailment, this discussion is only provided as one ailment that may be effectively treated by the various embodiments of the invention described herein.

It is known to provide the combination of two or more pharmaceutical formulations to treat weight loss and obesity. There exist methods of treating a patient suffering from obesity or a patient wanting to achieve weight loss, comprising the step of co-administering to the patient a therapeutically-effective amount of phentermine and metformin. In embodiments, this combined administration may be performed to: (1) promote weight loss; (2) increase weight reduction; and (3) decrease blood sugar levels. In embodiments, the combined administration is in the form of a medical treatment prescribed by a physician or any otherwise licensed individual and/or entity. In embodiments, the administration is the manufacturing of the medication in either separate dosages or combined dosages. There is also a process for preparing tablet dosage forms of poorly-compressible pharmaceutical agents. However, these treatments and processes are inadequate to effectively provide a mechanism for self-management and self-titration of medications.

In another example, it is known to provide the combination of three or more pharmaceutical formulations in different forms and arrangement. There exist methods of co-administering to the patient a therapeutically-effective amount of memantine, simvastatin, and Lisinopril. In embodiments, the combined administration may be performed to: (1) treat dementia associated with Alzheimer's disease; (2) lower LDL, “bad” cholesterol and fats and raise HDL, “good” cholesterol in the blood; and (3) lower blood pressure. Lastly, there exists an embodiment providing a layered pharmaceutical formulation comprising two or more pharmaceutical layers and an intermediate layer disposed between at least two of the two or more pharmaceutical layers. In some embodiments the intermediate layer is configured to dissolve in vivo to thereby leave the two or more pharmaceutical layers substantially intact, but physically separated, essentially forming two distinct pills. In some embodiments the dissolution rate of one of the separated two or more pharmaceutical layers is substantially similar to that of a singly compressed tablet comprising the same pharmaceutical composition as that of the pharmaceutical layer. Again, these treatments and processes are inadequate to effectively provide a mechanism for self-management and self-titration of medications.

Providing a combination of pharmaceutical agents to treat any physical condition, such as obesity or other ailments as described above, is known. However, the effective administration of these type of medications is limited in the physical composition of the pharmaceutical agents as well as their methods of administration.

### SUMMARY OF THE INVENTION

The present invention provides a versatile and effective manner to provide a single tablet or tablet arrangement or other medication delivery system for a combination of medications comprising of two or more active ingredients to be administered by severing along the score line to separate the active ingredients and to consume each active ingredient separately or in various ratios depending on the applicable pharmaceutical agent that a patient desires to ingest or otherwise administer. In some embodiments, a single tablet comprising of two or more active ingredients is administered by severing along one or more score lines to separate said active ingredients to consume two or more active ingredients in combination. Thus, this present invention provides a variety of alternatives to a consumer for administrating a combination of active ingredients while varying the relevant rations of the two or more active ingredients.

### BRIEF DESCRIPTION OF DRAWINGS

Other aspects of the disclosure will be readily apparent from the description below and the appended drawings, in which like reference numerals refer to similar parts throughout, which are meant to illustrate and not to limit the disclosure, and in which:

FIG. 1 illustrates an embodiment of a single tablet comprising of two active ingredients with one score line.

FIG. 1A illustrates an embodiment of a single tablet comprising of two active ingredients with one score line.

FIG. 1B illustrates an embodiment of a single tablet comprising of two active ingredients with one score line.

FIG. 2 illustrates an embodiment of a single tablet comprising of two active ingredients with two score lines.

FIG. 2′ illustrates an embodiment of a single tablet comprising of two active ingredients with two score lines and of which one active ingredient is severed off along said score lines.

FIG. 2A illustrates an embodiment of a single tablet comprising of two active ingredients with two intersecting score lines.

FIG. 2B illustrates an embodiment of a single tablet comprising of two active ingredients with two intersecting score lines.

FIG. 3 illustrates an embodiment of a matrix tablet with twenty equally-sized recessed rectangular panels arranged in a four panel by five panel with each panel having its own fracture score line within a large rectangle.

FIG. 3A illustrates an embodiment of a matrix tablet with six equally-sized recessed rectangular panels arranged in a two panel by three panel with each panel having its own fracture score line within a large rectangle.

FIG. 3B illustrates an embodiment of a matrix tablet with eight equally-sized recessed rectangular panels arranged in a two panel by four panel with each panel having its own fracture score line within a large rectangle.

FIG. 3C illustrates an embodiment of a linear tablet with three equally-sized rectangular panels arranged with two score lines.

FIG. 3D illustrates an embodiment of a matrix tablet with nine equally-sized recessed rectangular panels arranged in a three panel by three panel with each panel having its own fracture score line within a large rectangle.

FIG. 3E illustrates an embodiment of a matrix tablet with twelve equally-sized recessed rectangular panels arranged in a three panel by four panel with each panel having its own fracture score line within a large rectangle.

FIG. 4 illustrates an embodiment of a central hub formed a pharmaceutical composition with five peripheral extensions formed by pharmaceutical compositions connected to central hub by fracture lines.

FIG. 4A illustrates an embodiment of a central hub formed a pharmaceutical composition with five peripheral extensions formed by pharmaceutical compositions connected to central hub by five fracture lines.

FIG. 4B illustrates an embodiment of a central hub formed a pharmaceutical composition with six peripheral extensions formed by pharmaceutical compositions connected to central hub by six fracture lines.

FIG. 4C illustrates an embodiment of a central hub formed a pharmaceutical composition with seven peripheral extensions formed by pharmaceutical compositions connected to central hub by seven fracture lines.

FIG. 4D illustrates an embodiment of a central hub formed a pharmaceutical composition with eight peripheral extensions formed by pharmaceutical compositions connected to central hub by eight fracture lines.

FIG. 4E illustrates an embodiment of a central hub formed a pharmaceutical composition with three peripheral extensions formed by pharmaceutical compositions connected to central hub by three fracture lines.

FIG. 4F illustrates an embodiment of a central hub formed a pharmaceutical composition with four peripheral extensions formed by pharmaceutical compositions connected to central hub by four fracture lines.

FIG. 5 illustrates an embodiment of a three-dimensional cubic tablet with twenty-four score lines.

FIG. 6 illustrates an embodiment of a tablet with multiple pharmaceutical compositions held together by a connector for organization.

FIG. 7A illustrates an exemplary pill cutter according to this invention shown in the open position.

FIG. 7B illustrates an exemplary pill cutter according to this invention shown in the closed position.

FIG. 8A is a cross-sectional view of an inhaler according to an embodiment of the present invention.

FIG. 8B is a cross sectional view of the inhaler of FIG. 8A taken along line VIII-VIII of FIG. 8A.

FIG. 9 is a side view of a syringe according to an alternate embodiment of the present invention.

FIG. 9A is an enlarged view of the fluid cartridge having multiple fluid compartments for a syringe according to an alternate embodiment of the present invention.

FIG. 9B is a cross-sectional view of the fluid cartridge of FIG. 9A showing two separate fluid compartments.

FIG. 9C is a cross-sectional view of a fluid cartridge similar to FIG. 9B but having four different fluid compartments.

FIG. 9D illustrates an embodiment including two different dosage buttons in accordance with an embodiment of the present invention.

FIG. 9E illustrates an arrangement including four different dosage buttons in accordance with an embodiment of the present invention.

FIG. 9F illustrates an embodiment of the invention where a syringe utilizes two different fluid compartments for the cartridge member and the fluid path for the medications being delivered through the needle and to the patient.

### DETAILED DESCRIPTION OF INVENTION

The invention set forth herein provides a unique and convenient system, product and method for manufacturing and administering a wide variety of drugs and medications by way of a single pill arrangement comprising multiple medications that may be split or scored or otherwise distributed by the patient in a variety of different ratios for the relevant medications. Some of the benefits include the ability to customize drugs and medications prescribed by the doctor to use hourly, daily, weekly, or monthly. The ability to organize drugs and medications makes it easier for patients to keep track of the administration of the drugs and furthermore, it provides a convenient system for administering medications for example, during work or on vacation and during the weekend. The ability to organize and customize drugs and medications will make it easier for the patient to self-titrate and furthermore lessen the probability of taking the wrong medication by providing a visual indication of the mediation already taken during a prescribed time period. The invention set forth allows the patient to view all at once the drugs and medications needed for the day or the week, and determine which drugs and medications were already taken or need to be taken. In addition to the shape and arrangement of the subject pill, the invention may further utilize labelling of the pill or identification using colors or color coordination to more effectively inform the patient of the proper dosage and treatment to be taken.

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., at least one) of the grammatical object of the article. By way of example, “an element” means one element or more than one element.

The term “comprise” and “comprising” are used in the inclusive, open sense, meaning that additional elements may be included.

The term “including” is used to mean “including but not limited to.” “Including” and “including but not limited to” are used interchangeably.

The term “patient” refers to a mammal in need of a particular treatment. In a preferred embodiment, a patient is a primate, canine, feline, aquatic, equine, or cryonic. In another preferred embodiment, a patient is a human and is synonymous with a user.

The terms “co-administration” and “co-administering” refer both to concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “solvate” refers to a pharmaceutically acceptable form of a specified compound, with one or more solvent molecules, that retains the biological effectiveness of such compound. Examples of solvates include compounds of the invention in combination with solvents such, for example, water (to form the hydrate), isopropanol, ethanol, methanol, dimethyl sulfoxide, ethyl acetate, acetic acid, ethanolamine, or acetone etc. Also included are formulations of solvate mixtures such as a compound of the invention in combination with two or more solvents.

The present invention provides acceptable pharmaceutically compositions which comprise a pharmaceutical drug amount of two or more described. As described in detail below, the pharmaceutical compositions of the present invention may be specifically formulated for administration in solid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, gel, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a therapeutic agent in a composition of the present invention which is effective for producing some desired therapeutic effect in at least a subpopulation of cells in a patient and/or an animal at a reasonable benefit/risk ratio applicable to any medical treatment. The phrase “therapeutically-acceptable” is employed herein to refer to those compounds, materials, compositions, and/or dosage forms which are, within the scope of sound medical judgment, suitable for use in contact with the issues of human beings and animals without excessive toxicity, irritation, allergic response, or other problem or complication, commensurate with a reasonable benefit/risk ratio.

As set out above, certain embodiments of the compounds found in the present compositions may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds comprised in compositions of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the compounds that the present compositions comprise include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenyl acetic, glutamic, benzoic, salicyclic, sulfanilic, 2-acetoxybenzoic, fumaric, toluenesulfonic, methanesulfonic, ethane disulfonic, oxalic, isothionic, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredients which can be combined with a carrier material to produce a single dosage form will vary depending upon the host being treated, the particular mode of administration. The amount of active ingredients which can be combined with a carrier material to produce a single dosage form will generally be those amounts of the compounds which produce a therapeutic effect. Generally, out of one hundred percent, this amount will range from about 0.1 percent to about ninety-nine percent of active ingredients, from about 5 percent to about 70 percent, or from about 10 percent to about 30 percent.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredients. A composition of the present invention may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

A pharmaceutical composition may be configured in various ways. For example, a pharmaceutical composition may be configured in elliptical shapes, spherical shapes, cubic shapes, oblong shapes, square shapes or flat shapes. In some embodiments pharmaceutical formulations are combined with fillers or excipients and placed in tablets, granules or capsules for later administration. In some embodiments, the tablets are configured in spherical, elliptical, elongated, cubic, lenticular or capsule shapes.

For example, in some embodiments, a composition comprises a flat portion of a pharmaceutical formulation. In some embodiments, a composition comprises a rounded portion of a pharmaceutical formulation. In some embodiments, a layer comprises a conical section of a pharmaceutical formulation. In some embodiments, a composition comprises an elliptical section of a pharmaceutical formulation. In some embodiments, a composition comprises a sideways section of a pharmaceutical formulation. In some embodiments, a composition comprises a cubical section of a pharmaceutical formulation. In some embodiments, a composition comprises a wedge of a pharmaceutical formulation. In some embodiments, a composition comprises a substantial portion of a pharmaceutical formulation. A substantial portion is preferably at least about 25% of the pharmaceutical formulation and more preferably at least about 50% of the pharmaceutical formulation.

In certain embodiments, it may be advantageous for the pharmaceutical formulation to have a relatively large amount of the first pharmaceutical component compared to the second pharmaceutical component. In certain instances, the ratio of the first component to the second component is 12:1, 11:1, 10:1, 9:1, 8:1, 7:1, 6:1, or 5:1. In certain embodiments, it may be preferable to have a more equal distribution of pharmaceutical components. In certain instances, the ratio of the first component to the second component is 4:1, 3:1, 2:1, 1:1, 1:2, 1:3, or 1:4.

In certain embodiments, it may be advantageous for the pharmaceutical combination to have 6, 8, 10, 12, or 14 equally-sized recessed rectangular panels arranged in a 3, 4, 5, or 6 panel by 2 panel format with each panel having its own raised border within a large rectangle. In certain embodiments, it may be advantageous for the pharmaceutical combination to have 12, 15, 18, or 21 equally-sized recessed rectangular panels arranged in a 4, 5, 6, or 7 panel by 3 panel format with each panel having its own raised border within a large rectangle.

In certain embodiments, it may be advantageous for the pharmaceutical combination to have 6, 8, 10, 12, or 14 equally-sized recessed spherical panels arranged in a 3, 4, 5, or 6 panel by 2 panel format with each panel having its own raised border within a large rectangle. In certain embodiments, it may be advantageous for the pharmaceutical combination to have 12, 15, 18, or 21 equally-sized recessed spherical panels arranged in a 4, 5, 6, or 7 panel by 3 panel format with each panel having its own raised border within a large rectangle.

In some embodiments, at least one pharmaceutical composition reacts when brought into contact with another of the pharmaceutical composition within the layered pharmaceutical formulation. In some embodiments, at least one pharmaceutical composition does not react when brought into contact with another of the pharmaceutical composition.

In a solid dosage embodiment, a pharmaceutical composition includes two or more pharmaceutical components and a score disposed between at least two of the two or more pharmaceutical components.

The shape of the solid dosage form comprising at least one score may vary. For example, the solid dosage form may have, without limit, a substantially oval, ellipsoid, rectangular, cubic, discoid, circular, square, triangular, or hexagonal shape. The shape of the solid dosage form is such that the solid dosage form comprises an upper surface and a lower surface. The upper and/or lower surfaces may be substantially flat (planar). Alternatively, the upper and/or lower surfaces may be convex in that the central portion of the surface is raised relative to the peripheral edges of the surface. In some embodiments, the solid dosage form may comprise a circumferential (belly) band. Non-limiting examples of suitable solid dosage units include tablets, caplets, and pills. In exemplary embodiments, the solid dosage form is a tablet. The tablet may be, without limit, compressed, compacted, molded, pressed, monolithic, layered, multiparticle, extruded, uncoated, or coated.

The type of score on the solid dosage form may vary. For example, the score may be a standard score, a decreasing score, a standard protruding score, a pressure sensitive score, a cut through score, a short score, or a partial score. The shape of the score may vary. For instance, the score may be a V-shaped groove, a U-shaped groove, combinations, or variations thereof. The inner angle of the groove may range from about 30 degrees to about 150 degrees. In one embodiment, the groove may have a V-shape with an inner angle of about 90 degrees. The depth of the groove may also vary. In various embodiments, the depth of the groove may range from about 2% to about 30% of the total thickness of the solid dosage form.

In some embodiments, the solid dosage form may comprise one score, with the score being located on either the upper surface or the lower surface of the dosage form. Alternatively, the solid dosage form may comprise a first score on the upper surface and a second score on the lower surface. In embodiments in which the solid dosage form comprises a single score on either one or both of the surfaces, the score is centrally located such that splitting the scored solid dosage form yields two equal-sized solid dosage portions (or halves). Generally, the score is parallel to the shortest axis of the solid dosage form.

In other embodiments, the solid dosage form may comprise two or more scores on the upper and/or lower surfaces. For example, one or both of the surfaces may comprise two, three, four, five, six, seven, or more scores. In cases where the solid dosage form comprises two parallel scores on one or both of the surfaces, splitting the scored solid dosage form yields three equal-sized solid dosage portions (or thirds). In instances where the solid dosage form comprises two intersecting sores or three parallel scores on one or both of the surfaces, splitting the scored solid dosage form yields four equal-sized solid dosage portions (or quarters). Similarly, the score lines may be intersecting rather than parallel to define a matrix of dosage portions as will described in more detail below.

In general, the score of the solid dosage form is a functional score. A score is functional if the solid dosage form can be split manually (i.e., by hand) or mechanically (i.e., using a tablet splitter) into equal-sized solid dosage portions, with minimal loss of mass. The split solid dosage portions have similar weights and similar contents (i.e., each split solid dosage portion has an equivalent fraction of the active ingredient present in the intact solid dosage form).

The active ingredients that comprise a combination therapy may be administered together via a single dosage form or by separate administration of each active agent. In certain embodiments, the first and second therapeutic agents are administered in a single dosage form. In certain embodiments, the first, second, and third therapeutic agents are administered in a single dosage form which may be selectively split to change or alter the ratio of each therapeutic agent as will be described below. The agents may be formulated into a single tablet, pill, capsule, or solution for parenteral administration and the like.

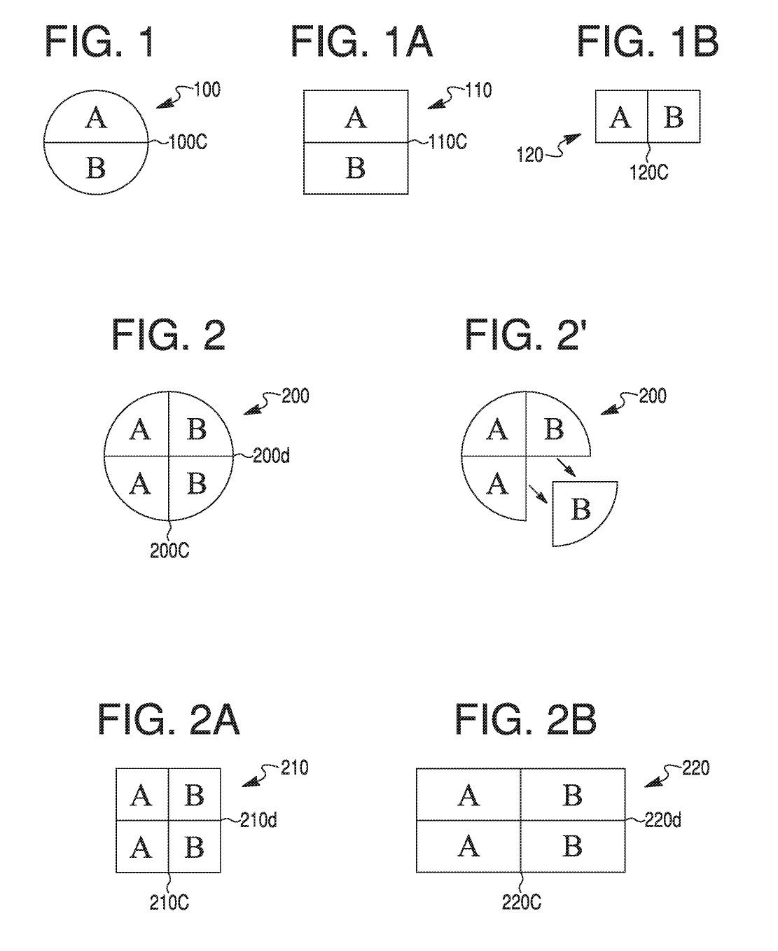

With reference to embodiment shown in FIG. 1, it will be understood by those skilled in the art that the single tablet shown in FIG. 1 comprises two active ingredients, A, B. In accordance with this invention, the tablet may be administered as a single dosage comprising 50% active ingredient A and 50% active ingredient B. Further in accordance with this invention, a user may choose to separate the tablet 100 into two separate components by severing along score line 100c, thereby separating active ingredient A from active ingredient B. In this instance, the user may choose to take only a single dose of active ingredient A. Alternatively, the user may choose to take only a single dose of active ingredient B. Thus, the present invention provides a variety of alternatives to a user for administrating a combination of active ingredient A and active ingredient B. In one case, the user may consume the entire tablet 100, thus, consuming 100% of active ingredient A and 100% of active ingredient B. In another case, the user may choose to only consume active ingredient A, thus consuming 100% of active ingredient A only. Alternatively, the user may choose to only consume active ingredient B, thus consuming 100% of active ingredient B only.

As clearly shown in FIG. 1, a pharmaceutical formulation 100 for the administration of two or more distinct active pharmaceutical ingredients, comprising a first pharmaceutical composition A, a second pharmaceutical composition B different from the first pharmaceutical composition A, and pharmaceutical compositions A and B being conjoined with fracture line 100c.

As clearly shown in FIGS. 1A and 1B, a pharmaceutical formulation 110 for the administration of two or more distinct active pharmaceutical ingredients, comprising a first pharmaceutical composition A, a second pharmaceutical composition B different from the first pharmaceutical composition A, and pharmaceutical compositions A and B being conjoined with fracture line 110c.

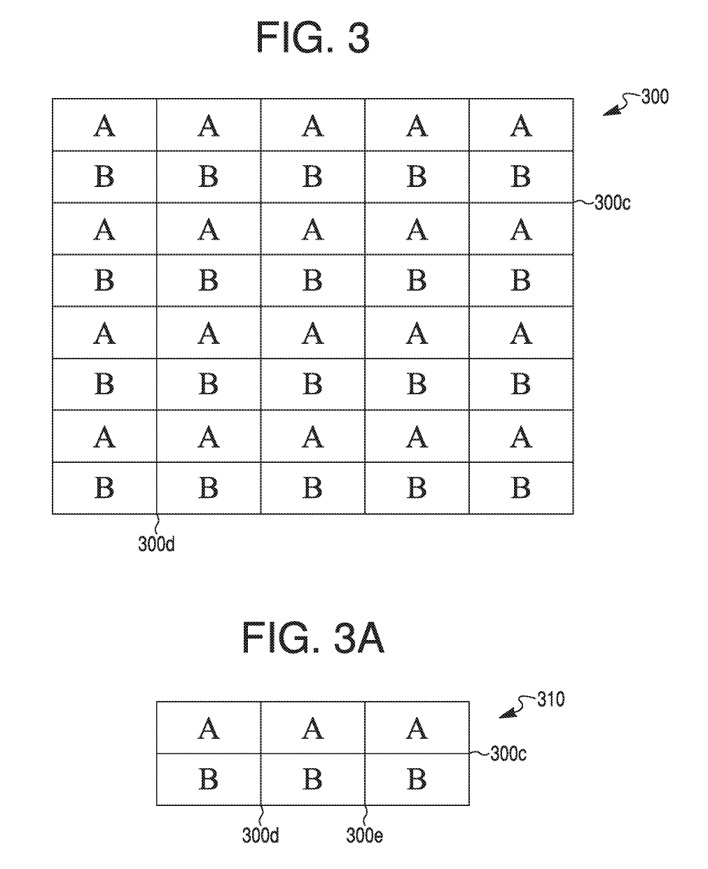

With reference to embodiment shown in FIG. 2, it will be understood by those skilled in the art that the single tablet shown in FIG. 2 comprises two active ingredients, A, B. In accordance with this invention, the tablet may be administered as a single dosage comprising 50% active ingredient A and 50% active ingredient B. In accordance with this invention, a user may choose to separate the tablet 200 into two separate components by severing along score line 200c, thereby separating active ingredient A from active ingredient B. In this instance, the user may choose to take only a single dose of active ingredient A. Alternatively, the user may choose to take only a single dose of active ingredient B. Thus, the present invention provides a variety of alternatives to a user for administrating a combination of active ingredient A and active ingredient B. In one case, the user may consume the entire tablet 200, thus, consuming 100% of active ingredient A and 100% of active ingredient B. In another case, the user may choose to only consume active ingredient A, thus consuming 100% of active ingredient A only. Further in accordance with this invention, a user may choose to separate tablet 200 into two separate components by severing along score line 200d, thereby separating active ingredients A and B from the other half of A and B. In this instance, the user may choose to take a single dosage of active ingredient A alone, active ingredient B alone, or active ingredients A and B together. Thus, the present invention provides a variety of alternatives to a user for administrating a combination of active ingredient A and active ingredient B.

Therefore, the structure of FIG. 2 provides at least one additional fracture line for separating said first pharmaceutical composition into fractional portions consisting of said first pharmaceutical composition. Furthermore, the structure of FIG. 2 provides plurality of fracture lines comprises forming at least two fracture lines that intersect with each other.

As clearly shown in FIG. 2, the formulation 200 comprises fracture line 200c and fracture line 200d that intersect each other. As clearly shown in FIG. 2A, the formulation 210 comprises facture line 210c and fracture line 210d that intersect each other. As clearly shown in FIG. 2B, the formulation 220 comprises fracture line 220c and 220d that intersect each other.

As clearly shown in FIG. 2, the formulation 200 comprises at least one additional fracture line 200d for separating first pharmaceutical composition A into fraction portions consisting of pharmaceutical composition A. The formulation comprises at least one additional fracture line 200d for separating second pharmaceutical composition B into fraction portions consisting of pharmaceutical compositions B.

As clearly shown in FIG. 2′, the formulation 200 comprises at least one additional fracture line 200d for separating first pharmaceutical composition A into fraction portions consisting of two pharmaceutical compositions A. The formulation comprises at least one additional fracture line 200d for separating second pharmaceutical compositions B into fraction portions consisting of two pharmaceutical compositions B. In total, four pharmaceutical compositions are formulated by said fracture lines which allows for the administration of an individual pharmaceutical composition B as clearly shown in FIG. 2′. This allows the user to self-titrate. For example, pharmaceutical composition A consists of metformin which is used to treat diabetes. Pharmaceutical composition B consists of phentermine which is used to treat obesity. If the user is feeling hypoglycemic, yet wants to lose weight, that patient can self-titrate by breaking the medications apart and by only digesting one of the phentermine sections.

As clearly shown in FIG. 2A, the formulation 210 comprises at least one additional fracture line 210d for separating first pharmaceutical composition A into fraction portions consisting of two pharmaceutical compositions A. The formulation comprises at least one additional fracture line 210d for separating second pharmaceutical composition B into fraction portions consisting of two pharmaceutical compositions B.

As clearly shown in FIG. 2B, the formulation 220 comprises at least one additional fracture line 220d for separating first pharmaceutical composition A into fraction portions consisting of two pharmaceutical compositions A. The formulation comprises at least one additional fracture line 220d for separating second pharmaceutical composition B into fraction portions consisting of two pharmaceutical compositions B.

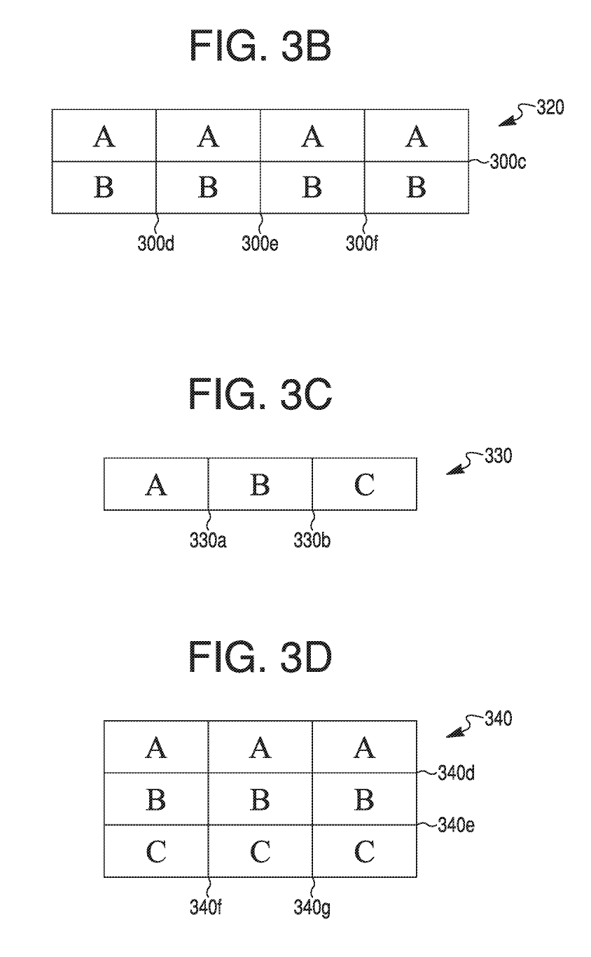

With reference to embodiment shown in FIG. 3, it will be understood by those skilled in the art that the single tablet shown in FIG. 3 comprises two active ingredients, A, B. In accordance with this invention, the tablet may be administered as a single dosage comprising 50% active ingredient A and 50% active ingredient B. In accordance with this invention, a user may choose to separate the tablet 300 into two separate components by severing along score line 300c, thereby leaving the row of active ingredients A and B from the rest of the row of active ingredients A and B. Then, a user may severe along the score line 300d in order to separate a single combination of the active ingredients A and B. In this instance, the user may choose to take only a single dose of active ingredient A. Alternatively, the user may choose to take only a single dose of active ingredient B. Thus, the present invention provides a variety of alternatives to a user for administrating a combination of active ingredient A and active ingredient B. In one case, the user may consumer the entire tablet 200, thus, consuming 50% of active ingredient A and 50% of active ingredient B. In another case, the user may choose to only consume active ingredient A, thus consuming 100% of active ingredient A only. Thus, the present invention provides a variety of alternatives to a user for administrating a combination of active ingredient A and active ingredient B. As an alternate embodiment, the ingredients A, B, C may be replaced with ingredients A, B, C, D, E, F, G, H aligned along a column of nine (9) rows and repeated as five (5) identical columns similar to FIGS. 3D and 3E.

Therefore, the structure of FIG. 3 includes first and second compositions as a matrix comprising a plurality of sections formed by said first and second pharmaceutical composition, and separating said plurality of sections by plural fracture lines.

As shown in FIG. 3A, the formulation 310 comprises pharmaceutical composition A and pharmaceutical composition B arranged as a matrix 310 comprising a plurality of sections formed by pharmaceutical composition A and pharmaceutical composition B, separated by plurality of fracture lines 310d and 310e.

As shown in FIG. 3B, the formulation 320 comprises pharmaceutical composition A and pharmaceutical composition B arranged as a matrix 320 comprising a plurality of sections formed by pharmaceutical composition A and pharmaceutical composition B, separated by plurality of fracture lines 310d, 310e and 310f.

As shown in FIG. 3C, pharmaceutical formulation 330 is formed with compositions A, B, and C are aligned in a linear arrangement with fracture line 330a separating composition A and composition B and fracture line 330b separating composition A and composition B. For example, pharmaceutical composition A consists of memantine which is used to treat dementia associated with Alzheimer's disease. Pharmaceutical B consists of simvastatin which is used to lower LDL, “bad” cholesterol and fats and raise HDL, “good” cholesterol in the blood. Pharmaceutical C consists of furosemide which is used to lower blood pressure. The user can self-titrate by breaking the medications apart and by only digesting pharmaceutical A, B, or C, or by taking A and B in the morning and C in the evening. Thus, the present invention provides a variety of alternatives to a user for administering a combination of active ingredients A, B, C.

As clearly shown in FIG. 3D, the formulation 340 comprises a third pharmaceutical composition C conjoined by fracture line 340f to pharmaceutical compositions A and B. For example, pharmaceutical composition A consists of memantine which is used to treat dementia associated with Alzheimer's disease. Pharmaceutical B consists of simvastatin which is used to lower LDL, “bad” cholesterol and fats and raise HDL, “good” cholesterol in the blood. Pharmaceutical C consists of furosemide which is used to lower blood pressure. If a patient is prescribed the combination of medications for just three times a week, the user can self-titrate by breaking the medications apart and by only digesting pharmaceutical A, B, or C, or by taking A and B in the morning and C in the evening. As clearly shown in FIG. 3E, the formulation 350 comprises a third pharmaceutical composition C conjoined by fracture line 350f to pharmaceutical compositions A and B. By way of example, the compositions A, B, C may be vitamins and the formulation 340 may be a three-day supply with each column A, B, C representing a different day of the week.

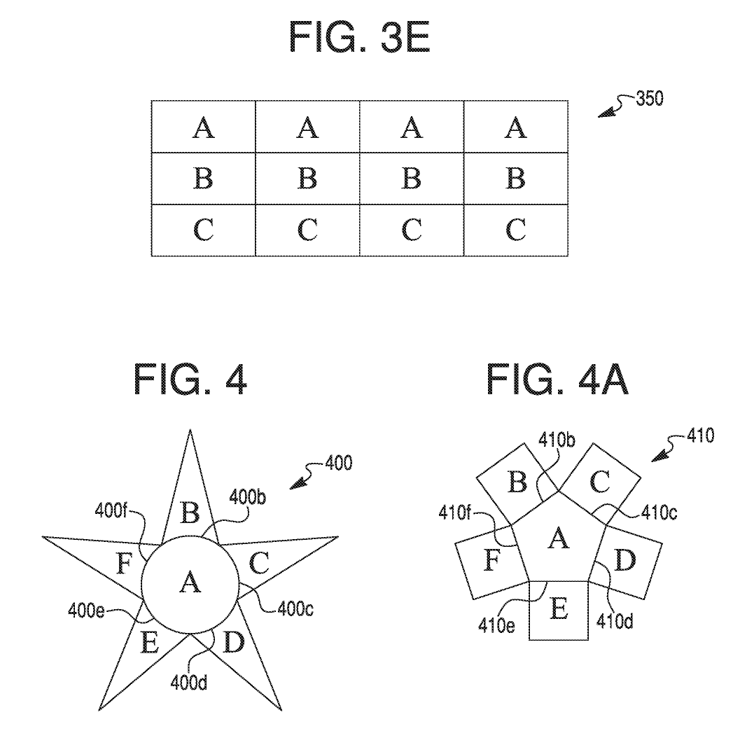

With reference to embodiment shown in FIG. 4, it will be understood by those skilled in the art that the single tablet shown in FIG. 4 comprises five active ingredients, A, B, C, D E. In accordance with this invention, a user may choose to take active ingredient B by severing along score line 400g, thereby leaving intact the rest of the embodiment and consuming active ingredient B. A user may choose to take active ingredient C by severing along score line 400h, thereby leaving intact the rest of the embodiment and consuming active ingredient C. A user may choose to take active ingredient D by severing along score line 400i, thereby leaving intact the rest of the embodiment and consuming active ingredient D. A user may choose to take active ingredient E by severing along score line 400j, thereby leaving intact the rest of the embodiment and consuming active ingredient E. A user may choose to take active ingredient F by severing along score line 400k, thereby leaving intact the rest of the embodiment and consuming active ingredient F. In these instances, the user may choose to take only a single dose of active ingredient A, B, C, D, E. Alternatively, the user may choose to take a combination of active ingredients A, B, C, D, E. Thus, the present invention provides a variety of alternatives to a user for administrating a combination of active ingredients A, B, C, D, E.

Therefore, the structure of FIG. 4 provides said first composition as a central hub of said formulation and forming said second pharmaceutical composition as a plurality of peripheral extensions extending from said central hub, and wherein each of said plurality of peripheral extensions is separated from said central hub by said at least one fracture line.

As clearly shown in FIG. 4A, the first pharmaceutical composition is formed a central hub A of the formulation 410 and the second pharmaceutical composition B is formed as a plurality of peripheral extensions of the central hub A. The peripheral extension B is separated from the central hub A by fracture line 410b. The third pharmaceutical composition is formed a central hub A of the formulation 410 and the third pharmaceutical composition C is formed as a plurality of peripheral extensions of the central hub A. The peripheral extension C is separated from the central hub A by fracture line 410c. The fourth pharmaceutical composition is formed a central hub A of the formulation 410 and the fourth pharmaceutical composition D is formed as a plurality of peripheral extensions of the central hub A. The peripheral extension D is separated from the central hub A by fracture line 410d. The fifth pharmaceutical composition is formed a central hub A of the formulation 410 and the fifth pharmaceutical composition D is formed as a plurality of peripheral extensions of the central hub A. The peripheral extension C is separated from the central hub A by fracture line 410d. The sixth pharmaceutical composition is formed a central hub A of the formulation 410 and the sixth pharmaceutical composition E is formed as a plurality of peripheral extensions of the central hub A. The peripheral extension E is separated from the central hub A by fracture line 410e. The seventh pharmaceutical composition is formed a central hub A of the formulation 410 and the seventh pharmaceutical composition F is formed as a plurality of peripheral extensions of the central hub A. The peripheral extension F is separated from the central hub A by fracture line 410f.

As clearly shown in FIG. 4B, the first pharmaceutical composition is formed a central hub A of the formulation 420 and the second pharmaceutical composition B is formed as a plurality of peripheral extensions of the central hub A. The peripheral extension B is separated from the central hub A by fracture line 420b. The third pharmaceutical composition is formed a central hub A of the formulation 420 and the third pharmaceutical composition C is formed as a plurality of peripheral extensions of the central hub A. The peripheral extension C is separated from the central hub A by fracture line 420c. The fourth pharmaceutical composition is formed a central hub A of the formulation 420 and the fourth pharmaceutical composition D is formed as a plurality of peripheral extensions of the central hub A. The peripheral extension D is separated from the central hub A by fracture line 420d. The fifth pharmaceutical composition is formed a central hub A of the formulation 420 and the fifth pharmaceutical composition E is formed as a plurality of peripheral extensions of the central hub A. The peripheral extension E is separated from the central hub A by fracture line 420e. The sixth pharmaceutical composition is formed a central hub A of the formulation 420 and the sixth pharmaceutical composition F is formed as a plurality of peripheral extensions of the central hub A. The peripheral extension F is separated from the central hub A by fracture line 420f. The seventh pharmaceutical composition is formed a central hub A of the formulation 420 and the seventh pharmaceutical composition F is formed as a plurality of peripheral extensions of the central hub A. The peripheral extension F is separated from the central hub A by fracture line 420f The eighth pharmaceutical composition is formed a central hub A of the formulation 420 and the eighth pharmaceutical composition G is formed as a plurality of peripheral extensions of the central hub A. The peripheral extension G is separated from the central hub A by fracture line 420g.

As shown in FIG. 4C, the first pharmaceutical composition is formed a central hub A of the formulation 430 and the second pharmaceutical composition B is formed as a plurality of peripheral extensions of the central hub A. The peripheral extension B is separated from the central hub A by fracture line 430b. The third pharmaceutical composition is formed a central hub A of the formulation 430 and the third pharmaceutical composition C is formed as a plurality of peripheral extensions of the central hub A. The peripheral extension C is separated from the central hub A by fracture line 430c. The fourth pharmaceutical composition is formed a central hub A of the formulation 430 and the fourth pharmaceutical composition D is formed as a plurality of peripheral extensions of the central hub A. The peripheral extension D is separated from the central hub A by fracture line 430d. The fifth pharmaceutical composition is formed a central hub A of the formulation 430 and the fifth pharmaceutical composition E is formed as a plurality of peripheral extensions of the central hub A. The peripheral extension E is separated from the central hub A by fracture line 430e. The sixth pharmaceutical composition is formed a central hub A of the formulation 430 and the sixth pharmaceutical composition F is formed as a plurality of peripheral extensions of the central hub A. The peripheral extension F is separated from the central hub A by fracture line 430f. The seventh pharmaceutical composition is formed a central hub A of the formulation 430 and the seventh pharmaceutical composition F is formed as a plurality of peripheral extensions of the central hub A. The peripheral extension F is separated from the central hub A by fracture line 430f The eighth pharmaceutical composition is formed a central hub A of the formulation 430 and the eighth pharmaceutical composition G is formed as a plurality of peripheral extensions of the central hub A. The peripheral extension G is separated from the central hub A by fracture line 430g. The ninth pharmaceutical composition is formed a central hub A of the formulation 430 and the eighth pharmaceutical composition H is formed as a plurality of peripheral extensions of the central hub A. The peripheral extension H is separated from the central hub A by fracture line 430h.

As shown in FIG. 4D, the first pharmaceutical composition is formed a central hub A of the formulation 440 and the second pharmaceutical composition B is formed as a plurality of peripheral extensions of the central hub A. The peripheral extension B is separated from the central hub A by fracture line 440b. The third pharmaceutical composition is formed a central hub A of the formulation 440 and the third pharmaceutical composition C is formed as a plurality of peripheral extensions of the central hub A. The peripheral extension C is separated from the central hub A by fracture line 440c. The fourth pharmaceutical composition is formed a central hub A of the formulation 440 and the fourth pharmaceutical composition D is formed as a plurality of peripheral extensions of the central hub A. The peripheral extension D is separated from the central hub A by fracture line 440d. The fifth pharmaceutical composition is formed a central hub A of the formulation 440 and the fifth pharmaceutical composition E is formed as a plurality of peripheral extensions of the central hub A. The peripheral extension E is separated from the central hub A by fracture line 440e. The sixth pharmaceutical composition is formed a central hub A of the formulation 440 and the sixth pharmaceutical composition F is formed as a plurality of peripheral extensions of the central hub A. The peripheral extension F is separated from the central hub A by fracture line 440f. The seventh pharmaceutical composition is formed a central hub A of the formulation 440 and the seventh pharmaceutical composition F is formed as a plurality of peripheral extensions of the central hub A. The peripheral extension F is separated from the central hub A by fracture line 440f The eighth pharmaceutical composition is formed a central hub A of the formulation 440 and the eighth pharmaceutical composition G is formed as a plurality of peripheral extensions of the central hub A. The peripheral extension G is separated from the central hub A by fracture line 440g. The ninth pharmaceutical composition is formed a central hub A of the formulation 440 and the ninth pharmaceutical composition H is formed as a plurality of peripheral extensions of the central hub A. The peripheral extension H is separated from the central hub A by fracture line 440h. The tenth pharmaceutical composition is formed a central hub A of the formulation 440 and the tenth pharmaceutical composition I is formed as a plurality of peripheral extensions of the central hub A. The peripheral extension I is separated from the central hub A by fracture line 440i. For example, pharmaceutical composition A consists of aspirin, pharmaceutical composition B consists of Vitamin B, pharmaceutical composition C consists of Vitamin C, pharmaceutical composition D consists of Vitamin D, pharmaceutical composition E consists of memantine, pharmaceutical composition F consists of simvastatin, pharmaceutical composition G consists of furosemide, pharmaceutical composition H consists of aspirin, and pharmaceutical composition I consists of aspirin. If the user would like to just take Vitamin C, the patient self-titrate by breaking off pharmaceutical composition C. If the user would like to take Vitamin A and B, the patient can self-titrate by breaking off pharmaceutical A and B and take them together. If the user would like to take memantine in the morning and take simvastatin and furosemide in the evening, the patient can self-titrate by breaking off pharmaceutical composition E in the morning and by breaking off pharmaceutical compositions F and G in the evening. If the user needs to aspirin in-between medications, the patient can break off pharmaceutical composition H or I, in addition to A.

As shown in FIG. 4E, the first pharmaceutical composition is formed a central hub A of the formulation 450 and the second pharmaceutical composition B is formed as a plurality of peripheral extensions of the central hub A. The peripheral extension B is separated from the central hub A by fracture line 450b. The third pharmaceutical composition is formed a central hub A of the formulation 450 and the third pharmaceutical composition C is formed as a plurality of peripheral extensions of the central hub A. The peripheral extension C is separated from the central hub A by fracture line 450c. The fourth pharmaceutical composition is formed a central hub A of the formulation 450 and the fourth pharmaceutical composition D is formed as a plurality of peripheral extensions of the central hub A. The peripheral extension D is separated from the central hub A by fracture line 450d.

As shown in FIG. 4F, the first pharmaceutical composition is formed a central hub A of the formulation 460 and the second pharmaceutical composition B is formed as a plurality of peripheral extensions of the central hub A. The peripheral extension B is separated from the central hub A by fracture line 460b. The third pharmaceutical composition is formed a central hub A of the formulation 460 and the third pharmaceutical composition C is formed as a plurality of peripheral extensions of the central hub A. The peripheral extension C is separated from the central hub A by fracture line 460c. The fourth pharmaceutical composition is formed a central hub A of the formulation 460 and the fourth pharmaceutical composition D is formed as a plurality of peripheral extensions of the central hub A. The peripheral extension D is separated from the central hub A by fracture line 460d. The fifth pharmaceutical composition is formed a central hub A of the formulation 460 and the fourth pharmaceutical composition E is formed as a plurality of peripheral extensions of the central hub A. The peripheral extension E is separated from the central hub A by fracture line 460e.

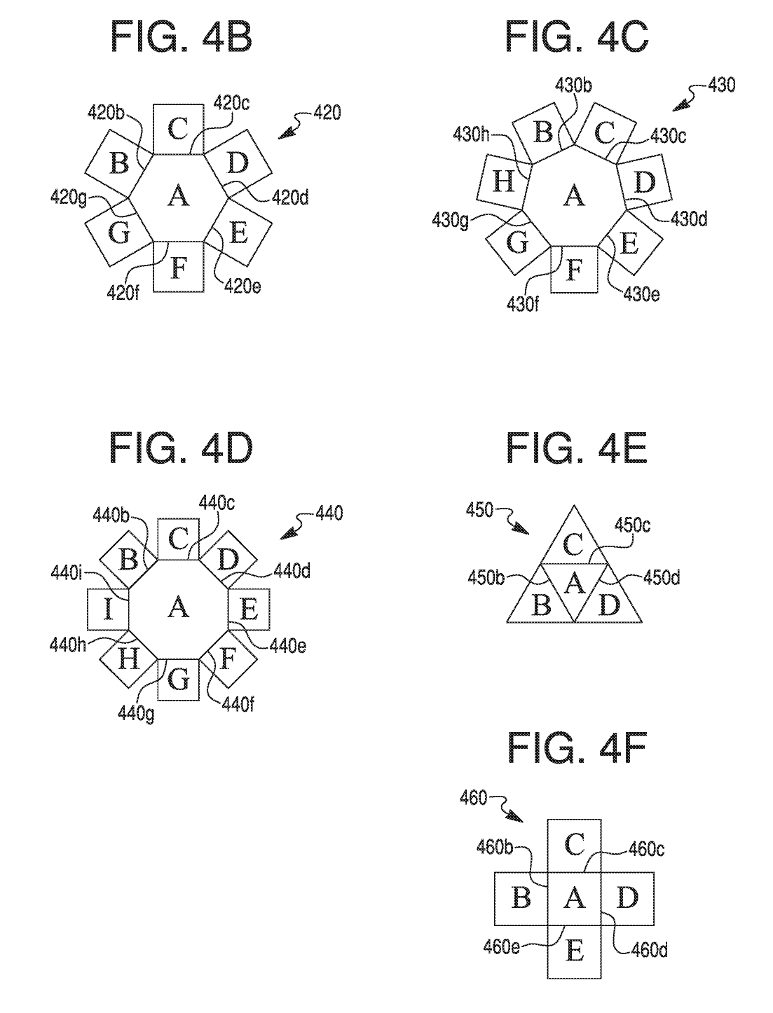

With reference to embodiment shown in FIG. 5, it will be understood by those skilled in the art that the single tablet shown in FIG. 5 comprises three active ingredients, A, B, C. In accordance with this invention, a user may choose to separate the tablet 500 in order to consume active ingredient A by severing along score line 500d, thereby leaving the layers of active ingredients B and C. Then, a user may severe along the score line 500f and 500g in order to separate a single dosage of active ingredient A. Alternatively, the user may choose to take only a double or triple dose of active ingredient A depending on the prescribed medication. In accordance with this invention, a user may choose to separate the tablet 500 in order to consume active ingredient B by severing along score line 500d and 500e, thereby leaving the layers of active ingredients A and C. Then, a user may severe along the score line 500f and 500g in order to separate a single dosage of active ingredient B. Alternatively, the user may choose to take only a double or triple dose of active ingredient B depending on the prescribed medication. In accordance with this invention, a user may choose to separate the tablet 500 in order to consume active ingredient C by severing along score line 500e, thereby leaving the layers of active ingredients A and B. Then, a user may severe along the score line 500f and 500g in order to separate a single dosage of active ingredient C. Alternatively, the user may choose to take only a double or triple dose of active ingredient C depending on the prescribed medication. Thus, the present invention provides a variety of alternatives to a user for administrating a combination of active ingredient A, B and C.

With reference to embodiment shown in FIG. 5, it will be understood by those skilled in the art that the single tablet shown in FIG. 5 comprises three active ingredients, A, B, C. In accordance with this invention, a user may choose to separate the tablet 500 in order to consume active ingredient A, B, C by severing along score line 500f, thereby leaving the other combined layers of active ingredients A, B, C. In one case, the user may consume the entire section of severed by 500f, thus, consuming 33.33% of active ingredient A, 33.33% of active ingredient B, and 33.33% of active ingredient C. In another case, the user may choose to only consume two active ingredients, A and B, thus consuming 50% of active ingredient A and 50% of active ingredient B. In another case, the user may choose to only consume active ingredient A, thus consuming 100% of active ingredient A only. Thus, the present invention provides a variety of alternatives to a user for administrating a combination of active ingredients A, B, C.

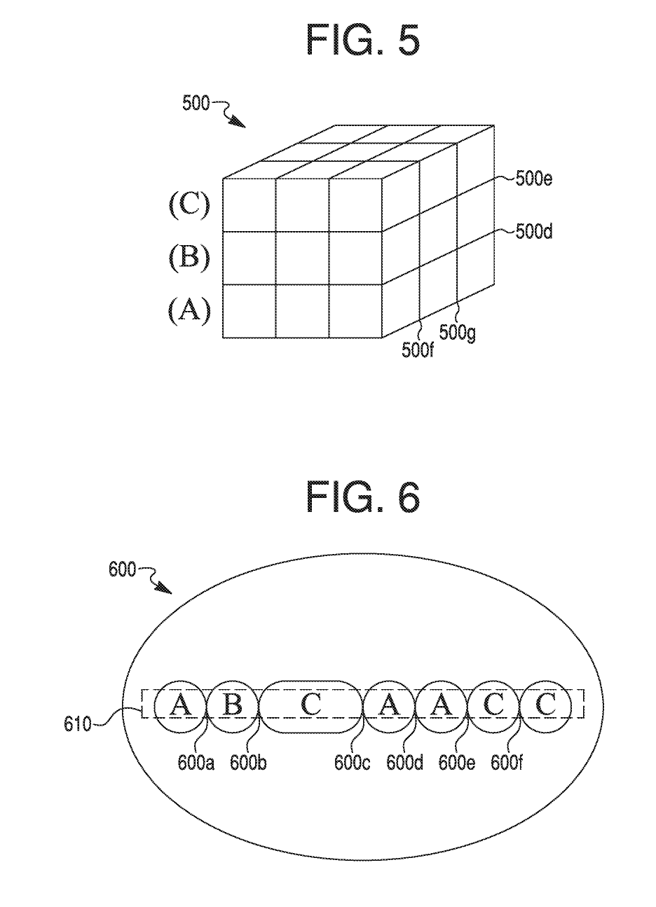

With reference to embodiment shown in FIG. 6, it will be understood by those skilled in the art that the single tablet shown in FIG. 6 comprises three active ingredients, A, B, C. In accordance with this invention, a user may choose to separate the tablet 600 separate components by severing along score line 600a, thereby separating active ingredient A from active ingredient B. In this instance, the user may choose to take only a single dose of active ingredient A. A user may also choose to separate the tablet 600 separate components by severing along score line 600b, thereby separating active ingredient B from active ingredient C. In this instance, the user may choose to take only a single dose of active ingredient A and active ingredient B. Alternatively, the user may choose to take only a single dose of active ingredient B. A user may also choose to separate the tablet 600 separate components by severing along score line 600c, thereby separating active ingredient C from active ingredient A. In this instance, the user may choose to take only a single dose of active ingredient A, active ingredient B or active ingredient C. Alternatively, the user may choose to take only a single dose of active ingredient A, B, or C. Thus, the present invention provides a variety of alternatives to a user for administrating a combination of active ingredient A, B, C. Managing medication is generally difficult for a user. The connector to keep all pharmaceutical compositions organized is valuable for a user who takes multiple medications in order to help increase patient convenience and patient compliance. Additionally, it is great for travel purposes because it is easy to carry. As shown in FIG. 6, the connector 610 is placed across the set of pharmaceutical compositions A, B, C in a linear fashion allowing the consumer to assemble and arrange certain medications in a convenient manner and to customize the drugs and medications for administration. The connector 610 may, for example, be made of a sticky strip that allows the consumer to choose needed and prescribed medications and place them directly onto the strip in order for the consumer to self-titrate in an organized and efficient manner. Other forms and arrangement of the connector 610 are also envisioned by the present invention.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredients therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredients only in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

As shown in FIG. 1, a method of manufacturing pharmaceutical formulation 100 of two or more distinct pharmaceutical ingredients with first active ingredient A and second active ingredient B different from active ingredient A, conjoined by providing fracture line 100c between pharmaceutical active ingredient A and pharmaceutical active ingredient B.

Another aspect of the present invention relates to a method of treating, by way of example, a patient suffering from obesity, comprising the step of co-administering to said patient a therapeutically effective amount of phentermine and metformin. Yet another aspect of the present invention relates to a method of achieving weight loss in a patient, comprising the step of co-administering to said patient a therapeutically effective amount of phentermine and metformin. In certain embodiments, an aforementioned method is practiced in conjunction or tandem with a medical procedure or the use of a medical device or both designed to contribute to the overall course of treatment.

A dissolution profile for a drug comprises the known dissolution rate and particular dissolution characteristics of the drug. A predictable dissolution profile for a specific drug allows for more accurate treatment of a given symptom.

In some embodiments, the two or more pharmaceutical lawyers comprise one or more immediate-release formulations. The term “immediate-release” is used herein to specify that the immediate release formulation is not configured to alter the dissolution profile of the pharmaceutical lawyer. For example, an immediate release pharmaceutical layer may be a pharmaceutical composition that does not contain ingredients included for the purpose of altering the dissolution profile. In some embodiments, the two or more pharmaceutical layers comprise one or more controlled-release formulations. The term “controlled-release” is used herein in its ordinary sense and thus includes pharmaceutical compositions combined with ingredients to later their dissolution profile. A “sustained-release” formulation is a type of controlled-release formulation, wherein ingredients have been added to a pharmaceutical composition such that the dissolution profile is extended over a longer period of time than that of an immediate release formulation comprising a similar pharmaceutical composition.

As noted above, pharmaceutical formulations may be configured in various shaped and sizes for ease of administration to a patient. Manufacture of pharmaceutical formulations configured in tablets comprises steps known in the art. For example, tablets may be prepared through wet-granulation, dry-granulation or direct compression. Layered pharmaceutical formulations may be configured in tablet form in a similar manner. To manufacture each pharmaceutical layer, one or more drugs are obtained in, for example, a crystalline, amorphous or powdered form, and mixed with or without diluents and/or excipients into a solid with pressure. The solid pharmaceutical layer is added with other pharmaceutical layers and/or intermediate layers and configured in a desired tablet geometry with pressure.

For methods of administering pharmaceutical compositions useful for affecting weight loss, suppressing appetite and/or treating obesity-related conditions in individuals controlled-release formulations help to suppress some if not all of the negative side effects that may arise from administration of such medication. Even in controlled-release formulations, however, the administration of certain anticonvulsants or opioid receptor antagonists at a full dosage may initially incur severe adverse side effects. Thus, at least initially, patients may be unable to tolerate a full dosage of the prescribed drug, which may include, but is not limited to an anticonvulsant or an opioid receptor antagonist. This intolerance may lead to more severe side effects and/or premature abandonment of the medication and/or the treatment program.

As shown in FIG. 1, a method of administrating pharmaceutical formulation 100 of two or more distinct pharmaceutical ingredients with first active ingredient A and second active ingredient B different from active ingredient A, conjoined by providing fracture line 100c between pharmaceutical active ingredient A and pharmaceutical active ingredient B, to alter ratio of pharmaceutical active ingredients A and B prior to ingestion.

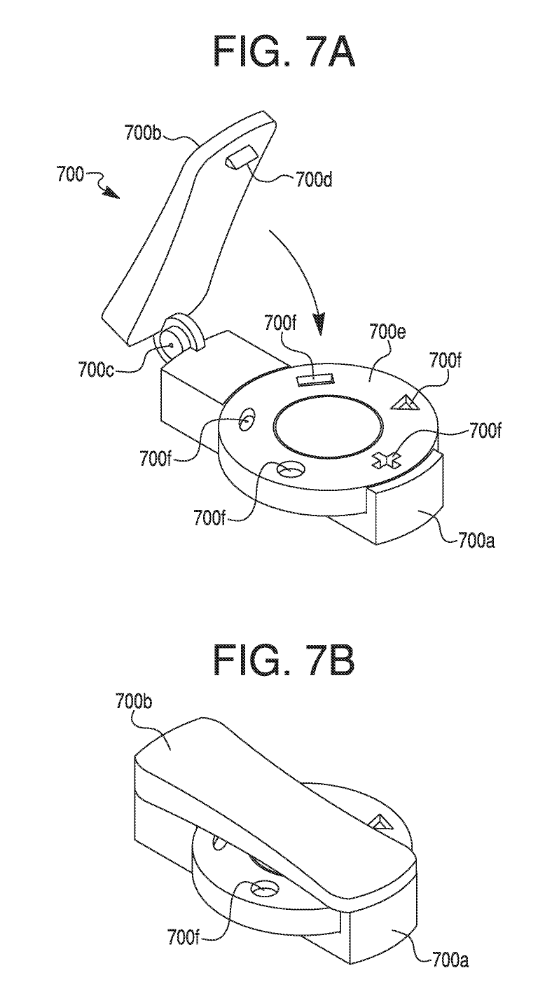

A multiple pill cutter provides an individual the ability to cut multiple pills simultaneously or near simultaneously, via one action from the individual (e.g. closing a lever or hinge, pressing a button, etc.). The multiple pill cutter can include a pill cutting bed on which pills can be placed. The multiple pill cutter can further include pill retainers that allow the individual to place and align multiple pills on the cutting bed. The pill cutting bed and the pill retainers can be housed within a lower portion. The multiple pill cutter can also include a top portion, one end of which is hinged to one end of the lower portion. An inner surface of the top portion can include a cutting blade for cutting pills placed on the cutting bed. The cutting blade is positioned such that when the top portion is lowered over the lower portion, the cutting edge of the cutting blade cuts through one or more pills placed on the cutting bed between the pill retainers. As clearly shown in FIG. 7A, the open-position pill cutter 700 can be employed for cutting different shape pills. The pill cutter 700 includes a base 700a and a top portion 700b affixed with a hinge at 700c. The base 700a is connected to a rotating cutting bed 700e which consists of different holes 700f in different shapes that allow different pills in different shapes to placed. In the arrangement show in in FIG. 7A, the holes 700f take the shape of various pills shown in FIGS. 1-4 herein. The different shapes include but are not limited to an oval, a triangle, a cross, a circle, and a rectangle. The top portion 700b includes a pill cutter bit placer 700d which holds different pill cutter bits which will cut into pills located in the oval, triangle, cross, circle, or rectangle shaped holes 700f in manner to remove the correct portions of the pills at issue in the manner described above. The patient places the medication composition into the hole 700f on the cutting bed 700e and presses down on the top portion 700b in order for the pill cutter bit placer 700d to cut the medication placed in the cutting bed 700e. FIG. 7B shows the movement of cutting the medication when the patient presses 700b down.

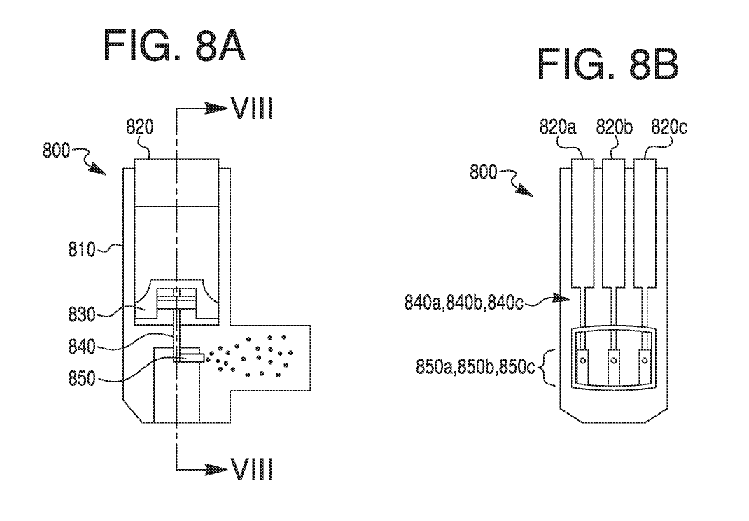

FIG. 8A is a cross-sectional view of an inhaler according to an embodiment of the present invention. As known by those of skill in the art, the inhaler 800 may include a housing 810 for containing a medication container 820 having an active drug, propellant and co-solvents as known in the art. Actuator seals 830 are provided for the metering valve 840 leading to the actuator nozzle 850. FIG. 8B is a cross sectional view of the inhaler of FIG. 8A taken along line VIII-VIII of FIG. 8A and showing three separate medication containers 820a, 820b, 820c leading to three distinct metering valves 840a, 840b, 840c. Three distinct actuator nozzles 850a, 850b, 850c are likewise provided for distributing a combination of three different medications in accordance with the present invention. The invention may provide a user with the ability to actuate one nozzle of the three nozzles 850a, 850b, 850c or the user may actuate all three nozzles or a combination of the three nozzles 850a, 850b, 850c to vary the distribution of the three medications contained in the medication containers 820a, 820b, 820c. As with other embodiments of the present invention, the inhaler 800 is designed to provide the user with the ability to administer different ratios of different medications. While the embodiment of FIG. 8A, 8B illustrates an device for three sets of medications, the present invention may encompass many different arrangements for distributing a variety of medications in a user-selected manner so that patients may self-manage and self-titrate—either simultaneously or in series.

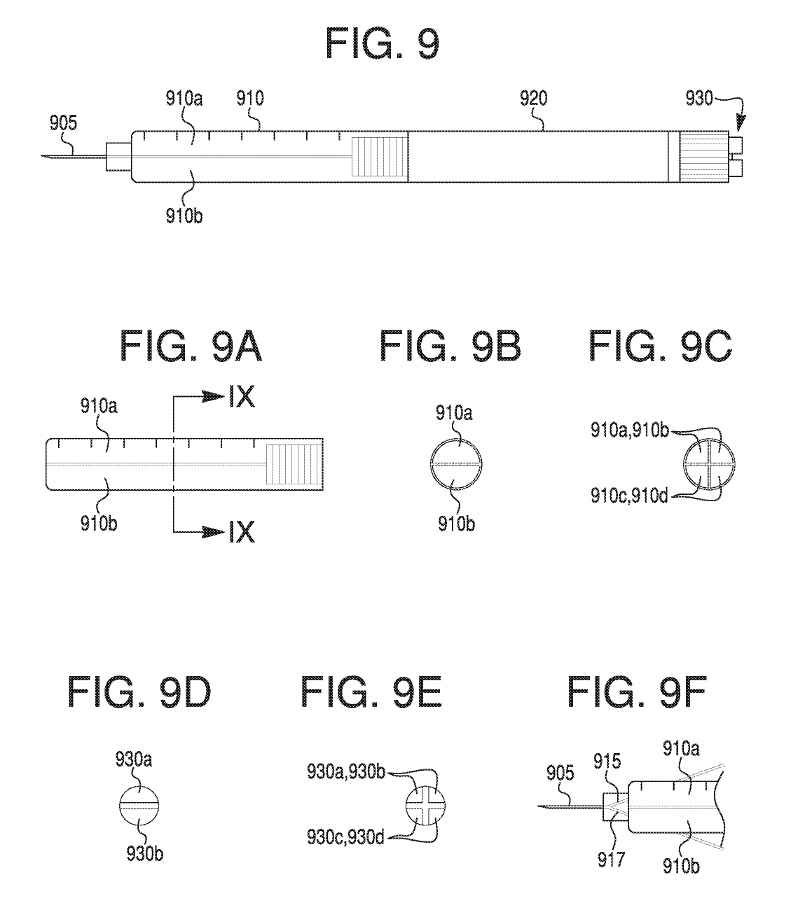

FIG. 9 is a side view of a syringe according to an alternate embodiment of the present invention. In accordance with the principals of the present invention, the syringe 900 is designed to selectively distribute and administer a variety of medications by selectively actuating a dosage button on the syringe 900. As known to those skilled in the art, the syringe 900 includes a needle, 905 a cartridge 910 and a plunger section 920 having dose button(s) 930 disposed thereon. In accordance with the invention, the fluid cartridge 910 is designed to contain and distribute multiple medications by utilizing multiple medication compartments 910a 910b which are individually actuated by multiple dosage button 930a, 930b (see FIG. 9D). FIG. 9A is an enlarged view of the fluid cartridge 910 having multiple fluid compartments 910a, 910b for a syringe according to an alternate embodiment of the present invention. FIG. 9B is a cross-sectional view of the fluid cartridge of FIG. 9A showing two separate fluid compartments 910a, 910b. FIG. 9C is a cross-sectional view of a fluid cartridge similar to FIG. 9B but having four different fluid compartments 910a, 910b, 910c, 910d. As described above, the actuator or dosage buttons 930 may be varied to provide a variety of different dosage arrangements for the patient. FIG. 9D illustrates an embodiment including two different dosage buttons 930a, 930b, and FIG. 9E illustrates an arrangement including four different dosage buttons 930a, 930b, 930c, 930d.

FIG. 9F illustrates an embodiment of the invention where a syringe utilizes two different fluid compartments 910a, 910b for the cartridge member 910 and the dual fluid path, including passageways 915, 917 for the medications being delivered from the compartments 910a, 910b through the needle 905 and to the patient. As described above with respect to the various embodiment of the present invention, the syringe 900 provides a delivery system for administering a variety of ratios of different medications so that the patient may self-manage and self-titrate the required medications at a given time. For example, the patient may wish to administer both basal insulin and GLP-1 using the syringe 900. By utilizing the two-part chamber 910, the user may inject basal insulin from compartment 910a and may inject GLP-1 from compartment 910b, wither simultaneously or in series using the dosage button 930a, 930b. Similarly, the patient may wish to administer PCSK9, basal insulin, GLP-1 and a placebo. By utilizing the embodiment of FIGS. 9C and 9E, the patient may utilize a four-part chamber 910 and administer all four of these medications from compartments 910a, 910b, 910c, 910d as shown in FIG. 9C by way of dosage button 930a, 930b, 930c, 930d. Other variations and arrangement will be apparent to those of skill in the art.

It will be appreciated by those skilled in the art that various modifications and changes can be made without departing from the scope of the disclosure. Such modifications and changes are intended to fall within the scope of the disclosure, as defined by the appended claims.

1. A pharmaceutical formulation for the administration of two or more distinct active pharmaceutical ingredients, comprising:
a first pharmaceutical composition;
a second pharmaceutical composition different from said first pharmaceutical composition, said first and second pharmaceutical compositions being conjoined; and
at least one fracture line disposed between said first and second pharmaceutical compositions.

2. The formulation according to claim 1, further comprising a third pharmaceutical composition conjoined with said first and second pharmaceutical compositions.

3. The formulation according to claim 1, wherein said first, second and third pharmaceutical compositions are aligned in a linear arrangement with fracture lines separates each of said first second and third pharmaceutical compositions.

4. The formulation according to claim 3, further comprising additional rows of said first, second and third pharmaceutical compositions arranged in parallel to said linear arrangement.

5. The formulation according to claim 1, wherein said at least one fracture line comprises a plurality of fracture lines.

6. The formulation according to claim 5, wherein said plurality of fracture lines comprises at least two fracture lines that intersect with each other.

7. The formulation according to claim 1, further comprising at least one additional fracture line for separating said first pharmaceutical composition into fractional portions consisting of said first pharmaceutical composition.

8. The formulation according to claim 1, wherein said first and second pharmaceutical compositions are arranged as a matrix comprising a plurality of sections formed by said first and second pharmaceutical composition, said plurality of sections being separated by plural fracture lines.

9. The formulation according to claim 1, wherein said first pharmaceutical composition is formed a central hub of said formulation and said second pharmaceutical composition is formed as a plurality of peripheral extensions of said central hub, and wherein each of said plurality of peripheral extensions is separated from said central hub by said at least one fracture line.

10. A method of manufacturing a pharmaceutical formation for the administration of two or more distinct active pharmaceutical ingredients, comprising:
forming a first pharmaceutical composition including a first active ingredient;
forming a second pharmaceutical composition including a second active ingredient different from said first active ingredient, said first and second pharmaceutical compositions being conjoined; and
forming at least one fracture line between said first and second pharmaceutical compositions.

11. The method according to claim 10, further comprising:
forming a third pharmaceutical composition conjoined with said first and second pharmaceutical compositions.

12. The method according to claim 10, further comprising:
aligning said first second and third pharmaceutical compositions in a linear arrangement and forming fracture lines between each of said first second and third pharmaceutical compositions.

13. The method according to claim 12, further comprising:
forming additional rows of said first, second and third pharmaceutical compositions arranged in parallel to said linear arrangement.

14. The method according to claim 10, wherein said forming said at least one fracture line comprises forming a plurality of fracture lines.

15. The method according to claim 14, wherein said forming said plurality of fracture lines comprises forming at least two fracture lines that intersect with each other.

16. The method according to claim 10, further comprising forming at least one additional fracture line for separating said first pharmaceutical composition into fractional portions consisting of said first pharmaceutical composition.

17. The method according to claim 10, wherein said forming said first and second pharmaceutical compositions comprises arranging said first and second compositions as a matrix comprising a plurality of sections formed by said first and second pharmaceutical composition, and separating said plurality of sections by plural fracture lines.

18. The method according to claim 10, wherein said forming said first pharmaceutical composition comprises forming said first composition as a central hub of said formulation and forming said second pharmaceutical composition as a plurality of peripheral extensions extending from said central hub, and wherein each of said plurality of peripheral extensions is separated from said central hub by said at least one fracture line.

19. A method of administering two or more distinct active pharmaceutical ingredients, comprising:
providing a first pharmaceutical composition including a first active ingredient;
providing a second pharmaceutical composition including a second active ingredient different from said first active ingredient, said first and second pharmaceutical compositions being conjoined; and
providing at least one fracture line between said first and second pharmaceutical compositions,
selectively separating said first and second pharmaceutical compositions along said at least one fracture line to alter a relative ratio of said first and second pharmaceutical compositions prior to ingestion.
